(19) United States
(12) Patent Application Publication
(10) Pub. No.: US 2014/0277211 A1
(43) Pub. Date: Sep. 18, 2014

(54) DEVICE FOR PERFORMING A SURGICAL PROCEDURE AND METHOD

(71) Applicant: KYPHON SARL, Neuchatel (CH)

(72) Inventors: AMY L. ARTHUR, Mountain View, CA (US); HESTER CHAN, Sunnyvale, CA (US)

(73) Assignee: KYPHON SARL, Neuchatel (CH)

(21) Appl. No.: 13/830,436

(22) Filed: Mar. 14, 2013

(51) Int. Cl. A61B 17/88

(52) U.S. Cl. CPC A61B 17/8855
USPC 606/86 R

(57) ABSTRACT

A device includes an inflatable member extending along an axis between a first end having a first opening and a second end having a second opening. An inner surface of the inflatable member defines a first passageway in the first end and a chamber in the second end. An inner member is disposed in the first passageway and extends through the first and second openings. The inner member includes an inner surface defining a second passageway. A first end of the inner member includes a first opening and a second end of the inner member has a second opening. The second end of the inflatable member is bonded to an outer surface of the inner member. A guide member is disposed in the second passageway and extends through the first and second openings of the inner member. Methods of use are provided.

### TECHNICAL FIELD

The present disclosure generally relates to medical devices for the treatment of musculoskeletal structures, and more particularly to a surgical system and method employing a cannulated inflatable bone tamp for fracture reduction.

### BACKGROUND

Height loss is commonly associated with fractures, such as, for example, spinal fractures, typically referred to as vertebral compression fractures. A large segment of osteoporotic patients experience vertebral compression fractures, with an estimated 700,000 such fractures occurring annually. Kyphoplasty is a minimally invasive procedure that is used to treat vertebral compression fractures using a combination of vertebroplasty utilizing a bone void filler, such as, for example, bone cement with balloon catheter technology. The kyphoplasty procedure restores height of the collapsed spinal bone which diminishes associated back pain.

Kyphoplasty procedures may also be used to treat fractures in other areas of a patient's body, such as, for example, a distal radius of the patient, such as, for example Colles' fractures. To treat a distal radius fracture using a kyphoplasty procedure, an inflatable bone tamp (IBT) is utilized. The IBT is used to percutaneously reduce comminuted, articular depressions in a controlled manner. Fracture morphologies, such as, for example, “die-punch” fractures are especially suited for correction by an IBT. IBTs are deployed to a surgical site, such as, for example, a bone defect through a working cannula. IBTs create well-defined voids. After the void is created by the IBT, the IBT is removed from the cannula and a material, such as, for example a bone void filler is delivered through the cannula and into the void. The bone void filler may be used in conjunction with percutaneous pins, ex-fixes, plates and/or screws for fracture fixation.

In conventional kyphoplasty procedures, the IBT is inserted adjacent the bone defect by inserting the IBT through a cannula. An inflatable member of the IBT is expanded to create a void in or adjacent the bone defect. After the IBT creates the void, the IBT is removed from the cannula and a bone void filler is delivered through the cannula to the void in order to at least partially fill the void. This disclosure describes an improvement over these prior art technologies.

### SUMMARY

Accordingly, a surgical system and method for correction of a bone injury or disorder are provided. In one embodiment, in accordance with the principles of the present disclosure, the surgical system includes a device for performing a surgical procedure. The device comprises an inflatable member extending along a longitudinal axis between a first end having a first opening and a second end having a second opening. An inner surface of the inflatable member defines a first passageway in the first end and a chamber in the second end. The first and second openings are in communication with the first passageway. An inner member is disposed in the first passageway and extends through the first and second openings. The inner member includes an inner surface defining a second passageway. A first end of the inner member includes a first opening and a second end of the inner member has a second opening. The first and second openings of the inner member are in communication with the second passageway. The second end of the inflatable member is bonded to an outer surface of the inner member. A guide member is disposed in the second passageway and extends through the first and second openings of the inner member.

In one embodiment, in accordance with the principles of the present disclosure, the surgical system includes a device comprising an inflatable member extending along a longitudinal axis between a first end having a first opening and a second end having a second opening. An inner surface of the inflatable member defines a first passageway in the first end and a chamber in the second end. The first and second openings are in communication with the first passageway. An inner member is disposed in the first passageway and extends through the first and second openings. The inner member includes an inner surface defining a second passageway. A first end of the inner member includes a first opening and a second end of the inner member has a second opening. The first and second openings of the inner member are in communication with the second passageway. The second end of the inflatable member is bonded to an outer surface of the inner member. A guide member is disposed in the second passageway and extends through the first and second openings of the inner member. An outer member comprises an inner surface defining a third passageway. The third passageway comprises a first lumen having the inflatable member disposed therein and a second lumen having a bone filler material or a tool configured to deliver bone filler material disposed therein. The first lumen is spaced apart from the second lumen. At least one of the first and second lumens have an aperture including a first portion extending parallel to the longitudinal axis and a second portion extending transverse to the longitudinal axis. The inflatable member is movable between a first orientation in which the chamber has a first diameter and a second orientation in which the chamber has a second diameter, the second diameter being greater than the first diameter.

In one embodiment, in accordance with the principles of the present disclosure, a method for correction of a bone injury is provided. The method comprises the steps of: providing a device comprising: an inflatable member extending along a longitudinal axis between a first end having a first opening and a second end having a second opening, an inner surface of the inflatable member defining a first passageway in the first end and a chamber in the second end, the first and second openings being in communication with the first passageway, an inner member disposed in the first passageway and extending through the first and second openings, the inner member including an inner surface defining a second passageway, a first end of the inner member including a first opening and a second end of the inner member having a second opening, the first and second openings of the inner member being in communication with the second passageway, the second end of the inflatable member being bonded to an outer surface of the inner member, and a guide member disposed in the second passageway and extending through the first and second openings of the inner member; creating an access path to a defect in a bone; inserting the guide member through the access path such that a distal end of the guide member engages tissue to fix the guide member relative to the bone; inserting the inner member over the guide member such that the chamber is positioned in or adjacent the defect; and delivering a material through the first passageway and into the chamber to move the inflatable member from a first orientation in which the chamber has a first diameter to a second orientation in which the chamber has a second diameter, the second diameter being greater than the first diameter such that the chamber creates a void in or adjacent the defect when the inflatable member is in the second orientation.

### BRIEF DESCRIPTION OF THE DRAWINGS

The present disclosure will become more readily apparent from the specific description accompanied by the following drawings, in which:

FIG. 1 is a side, cross sectional view of one embodiment of a surgical system in accordance with the principles of the present disclosure;

FIG. 2 is a side view of one embodiment of components of the system shown in FIG. 1 in accordance with the principles of the present disclosure;

FIG. 3 is a breakaway side, cross sectional view of components of the system shown in FIG. 1 positioned within a bone;

FIG. 4 is a side view of one embodiment of components of the system shown in FIG. 1 in accordance with the principles of the present disclosure;

FIG. 5 is a cross sectional view of the inflatable member shown in FIG. 5 taken along lines B-B;

FIG. 6A is a side, cross sectional view of a bone with a bone defect;

FIG. 6B a side, cross sectional view of the bone shown in FIG. 7A with a void created by the inflatable member shown in FIG. 5;

FIG. 7 is a side view of one embodiment of an inflatable member of the system shown in FIG. 1 in accordance with the principles of the present disclosure;

FIG. 8 is a cross sectional view of the inflatable member shown in FIG. 8 taken along lines C-C;

FIG. 9A is a side, cross sectional view of a bone with a bone defect;

FIG. 9B a side, cross sectional view of the bone shown in FIG. 10A with a void created by the inflatable member shown in FIG. 8;

FIG. 10 is a side, cross sectional view of one embodiment of a surgical system in accordance with the principles of the present disclosure;

FIG. 11 is a side, cross sectional view of a component of the system shown in FIG. 10;

FIG. 12 is a breakaway side, cross sectional view of the component shown in FIG. 11 positioned within a bone;

FIG. 13 a side, cross sectional view of the bone shown in FIG. 12 with voids created by the component shown in FIG. 11;

FIG. 14 is a breakaway side, cross sectional view of the component shown in FIG. 11 positioned within one of the voids shown in FIG. 13;

FIG. 15 is side, cross sectional view of the bone shown in FIG. 12 with voids created by the inflatable member shown in FIG. 11;

FIG. 16 is a breakaway side, cross sectional view of the component shown in FIG. 11 positioned within one of the voids shown in FIG. 15;

FIG. 17 is side, cross sectional view of the bone shown in FIG. 12 with voids created by the component shown in FIG. 11;

FIG. 18 is a side view of one embodiment of a component of a surgical system in accordance with the principles of the present disclosure; and

FIG. 19 is a top, cross sectional view of the component shown in FIG. 18.

Like reference numerals indicate similar parts throughout the figures.

### DETAILED DESCRIPTION

The exemplary embodiments of the surgical system and related methods of use disclosed are discussed in terms of medical devices for the treatment of musculoskeletal disorders and more particularly, in terms of a surgical system and method for bone repair. It is envisioned that the surgical system and method may be employed in applications such as for correction of fractures, depressions and breaks. For example, the surgical system and method can include an inflatable bone tamps comprising an inflatable member having various cross sectional configurations adapted to match the configuration of a bone defect, such as, for example, a fracture in a bone, such as, for example, a distal radius of a human patient. That is, the inflatable member, when in an expanded configuration, has a volume that is equal to or exceeds the volume of the bone defect such that the inflatable member can create a single void that consumes the entire bone defect. It is envisioned that this configuration is advantageous over inflatable members that create many small voids to consume one bone defect.

In one embodiment, the inflatable member has a cylindrical cross sectional configuration when the inflatable member is an expanded configuration. In one embodiment, the inflatable member has an asymmetrical cross sectional configuration when the inflatable member is an expanded configuration. In one embodiment, the inflatable member has a triangular cross sectional configuration when the inflatable member is an expanded configuration.

In one embodiment, the inflatable member having a selected cross sectional configuration is employed in a method for treating a bone defect, such as, for example a fracture, by inserting the inflatable bone member in or adjacent to the bone defect at a first selected trajectory relative to the bone and moving the expandable member to an expanded configuration such that the inflatable member creates a first void in the bone. The inflatable member is then removed from the first void and/or the bone and is inserted into the bone at a second selected trajectory relative to the bone. The inflatable member is then moved to the expanded configuration such that the inflatable member creates a second void in the bone. It is envisioned that the second void may overlap at least a portion of the first void and that the first and second voids may have a combined volume that is equal to or exceeds the volume of the bone defect such that the first and second voids consume the entire bone defect.

In one embodiment, the inflatable member has a first lobe and a second lobe that is spaced apart from the first lobe when the inflatable member is an expanded configuration. It is envisioned that the first lobe is inflatable is inflatable/deflatable independent of the second lobe, and vice versa. That is, the first lobe can have an expanded configuration while the second lobe has an unexpanded configuration and the second lobe can have an expanded configuration while the first lobe has an unexpanded configuration. In one embodiment, a multiple lumen catheter is used such that each lobe has access to its own lumen such that each lobe can be selectively inflated by delivering fluid through a respective lumen.

In one embodiment, an inflatable member having first and second lobes is employed in a method for treating a bone defect, such as, for example a fracture, by inserting the inflatable bone member in or adjacent to the bone defect and moving the expandable member to an expanded configuration such that the first lobe creates a first void and the second lobe creates a second void.

In one embodiment, the expandable member is moved relative to the bone such that the second lobe is positioned in the first void. The inflatable member is then moved to the expanded configuration such that the second lobe creates a third void adjacent the first void while the first lobe engages the first void to anchor the inflatable member relative to the bone. It is envisioned that the second lobe may be moved to the expanded configuration when the second lobe is positioned in the first void prior to moving the first lobe to the expanded configuration to create the third void such that the second lobe acts as to anchor the inflatable member relative to the bone while the third void is created.

In one embodiment, the expandable member is moved relative to the bone such that the first lobe is positioned in the second void. The inflatable member is then moved to the expanded configuration such that the second lobe creates a third void adjacent the second void while the first lobe engages the second void to anchor the inflatable member relative to the bone. It is envisioned that the first lobe may be moved to the expanded configuration when the first lobe is positioned in the second void prior to moving the second lobe to the expanded configuration to create the third void such that the first lobe acts as to anchor the inflatable member relative to the bone while the third void is created.

In one embodiment, the system includes a cannula including a first passageway including a first lumen having an inflatable member disposed therein and a second lumen having a bone filler material disposed therein. The first and second lumens each have a cylindrical cross sectional configuration and are spaced apart from one another. The inflatable member is moved through the first lumen such that at least a portion of the inflatable member is disposed adjacent a bone defect, such as, for example a fracture in a bone of a patient, such as, for example, a Colles fracture or distal radius fracture in a human patient. The inflatable member is moved to an expanded configuration such that the inflatable member creates a void in or adjacent the bone defect. The inflatable member is then moved to an unexpanded configuration and at least a portion of the inflatable member is retracted into the first lumen. The cannula is then rotated relative to the bone defect such that the second lumen is positioned in or adjacent the void. The bone filler material is then delivered through the second lumen and into the void to at least partially fill the void.

It is contemplated that one or all of the components of the surgical system may be disposable, peel-pack, pre-packed sterile devices. One or all of the components of the surgical system may be reusable. The surgical system may be configured as a kit with multiple sized and configured components, such as, for example, inflatable members (balloons) that are preformed to have different sizes and shapes.

It is envisioned that the present disclosure may be employed to treat bones, and in particular arm bones such as a distal radius. It should be understood that the present principles are applicable to any bone structures, including but not limited to bones of the spine, legs, feet, arms, etc. It is contemplated that the present disclosure may be employed with other osteal and bone related applications, including those associated with diagnostics and therapeutics. It is further contemplated that the disclosed surgical system and methods may alternatively be employed in a surgical treatment with a patient in a prone or supine position, and/or employ various surgical approaches, including anterior, posterior, posterior mid-line, direct lateral, postero-lateral, antero-lateral, etc. approaches in the calcaneus, spine or other body regions. The present disclosure may also be alternatively employed with procedures for treating the muscles, ligaments, tendons or any other body part. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The following disclosure includes a description of a surgical system for treating bone defects, such as, for example, fractures, including a cannulated inflatable bone tamp for fracture reduction. The disclosure also includes a description of related methods of employing the cannulated inflatable bone tamp. Alternate embodiments are also disclosed. Reference will now be made in detail to the exemplary embodiments of the present disclosure, which are illustrated in the accompanying figures. Turning now to FIGS. 1-19 there are illustrated components of a surgical system, such as, for example, a surgical system 30 and embodiments in accordance with the principles of the present disclosure.

The components of system 30 can be fabricated from biologically acceptable materials suitable for medical applications, including metals, synthetic polymers, ceramics and bone material and/or their composites, depending on the particular application and/or preference of a medical practitioner. For example, the components of system 30, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of balloon system 20 may have material composites, including the above materials, to achieve various desired characteristics such as strength, rigidity, elasticity, compliance, biomechanical performance, durability and radiolucency or imaging preference. The components of system 30, individually or collectively, may also be fabricated from a heterogeneous material such as a combination of two or more of the above-described materials. The components of system 30 may be monolithically formed, integrally connected or include fastening elements and/or instruments, as described herein.

System 30 is employed, for example, with an open, mini-open or minimally invasive surgical technique to move or apply pressure to a bone fragment, fracture or surface, such as, in treating distal radius fractures. System 30 includes an inflatable bone tamp, such as, for example, an inflatable member 40 extending along a longitudinal axis A between a first end 42 and a second end 44. An inner surface 46 of member 40 defines a passageway 48 extending through end 42 and a chamber 50 in end 44. Passageway 48 extends parallel to axis A, has a cylindrical cross sectional configuration and a uniform diameter along the length of passageway 48. It is envisioned that passageway 48 may be disposed in orientations relative to axis A, such as, for example, transverse, perpendicular and/or other angular orientations such as acute, obtuse, co-axial and/or may be offset or staggered. It is further envisioned that all or only a portion of passageway 48 may be variously configured and dimensioned, such as, for example, planar, concave, convex, hemispherical, polygonal, irregular, uniform, non-uniform, staggered, tapered, consistent or variable, depending on the requirements of a particular application.

Passageway 48 is in communication with chamber 50 such that a material, such as, for example, saline, a contrast solution or compressed air may be inserted through passageway 48 and into chamber 50 to move chamber 50 from a first unexpanded orientation in which chamber 50 has a first diameter to an second expanded orientation in which chamber 50 has a second diameter that is greater than the first diameter.

It is envisioned that chamber 50 can define a single or multi-layered balloon where each balloon layer has the same diameter and/or wall thickness, is comprised of the same material or materials having substantially identical mechanical properties, and has the same degree of molecular orientation in the body portion of the balloon. It will be apparent that in some situations it will be desirable to have some balloon layers having different thicknesses, materials, and/or degree of molecular orientations upon deflation, while at the same time having equivalent size, mechanical properties, and/or orientation upon inflation or expansion. For other applications, it will be apparent that one can vary size, material, and/or orientation to at least some degree, depending upon the requirements of a particular application.

It is contemplated that chamber 50 may be defined by an impenetrable structural layer having low friction surfaces so as to facilitate deployment of member 40 through a surgical instrument, such as, for example, a working cannula and prevent rupture of chamber 50 as it is inflated or expanded in situ. Further variations are contemplated involving different combinations of lubricating layers and structural layers. In some embodiments, structural layers of chamber 50 can contain polyamides, polyesters, polyethylenes, polyurethanes, their co-polymers and combinations thereof.

It is envisioned that chamber 50 can be adapted to withstand the particular stresses, pressures, and deformities to which they might be placed under when inflated or expanded within a surgical site, such as, for example, a bone void in one or more vertebrae. For example, because a top (outer) layer of chamber 50 may be exposed to sharp objects (such as calcified plaque, bone, bone spurs, or other natural protrusions within a patient's body), the top layer could be made from a more compliant material that is scratch and puncture resistant, than the layer or layers below the top layer (inner layer(s)). That is, the top or outer layer is made from a more compliant material that is scratch and puncture resistant and the inner layers of the multi-layer balloon, which are generally not exposed to sharp objects, made from a less compliant material with a higher burst strength. It is contemplated that further variations are possible, depending on which stresses, pressures, and deformities the layers must withstand in a particular medical application.

End 42 includes a first circular opening 52 that is in communication with passageway 48 and end 44 includes a second circular opening 54 that is in communication with chamber 50. Openings 52, 54 each extend parallel to axis A. It is contemplated that opening 52 and/or opening 54 may be disposed at alternate orientations, relative to axis A, such as, for example, transverse, perpendicular and/or other angular orientations such as acute or obtuse, co-axial and/or may be offset or staggered. It is further envisioned that all or only a portion of opening 52 and/or opening 54 may have alternate cross section configurations, such as, for example, oval, oblong, triangular, square, polygonal, irregular, uniform, non-uniform, offset, staggered, and/or tapered.

In one embodiment, shown in FIG. 1, chamber 50 has a substantially rectangular configuration when chamber 50 is in the second orientation. In one embodiment, shown in FIG. 2, chamber 50 has a bulbous configuration when chamber 50 is in the second orientation. It is envisioned that all or only a portion of chamber 50 may be variously configured and dimensioned, such as, for example, oval, oblong, triangular, square, polygonal, planar, concave, convex, hemispherical, polygonal, irregular, uniform, non-uniform, staggered, tapered, consistent or variable when chamber 50 is in the second orientation, depending on the requirements of a particular application.

In one embodiment, shown in FIG. 4, chamber 50 has an asymmetrical cross sectional configuration when chamber 50 is in the second orientation. That is, a first side of chamber 50 has a diameter that is different from a diameter of an opposite second side of chamber 50. In one embodiment, chamber 50 is formed to have an asymmetrical configuration by providing tubing with a uniform cross-section, such as, for example, a circular cross-section, and molding the tubing with an asymmetric mold. In one embodiment, chamber 50 is formed to have an asymmetrical configuration by extruding tubing having an asymmetric cross-section. In one embodiment, chamber 50 is formed to have an asymmetrical configuration by providing tubing with non-uniform material properties that may be molded within an asymmetric mold, such as, for example, providing tubing having a uniform or asymmetric cross-section, with one side of the tubing being softer than an opposite side of the tubing.

In one embodiment, shown in FIG. 7, chamber 50 has a triangular configuration when chamber 50 is in the second orientation. In one embodiment, chamber 50 is formed to have a triangular configuration by providing tubing with a uniform cross-section, such as, for example, a circular cross-section, and molding the tubing with a triangular mold. In one embodiment, chamber 50 is formed to have a triangular configuration by extruding tubing having a triangular cross-section. In one embodiment, chamber 50 is formed to have a triangular configuration by providing tubing with non-uniform material properties that may be molded within an asymmetric mold, such as, for example, providing tubing having a uniform or triangular cross-section, with at least one side of the tubing being softer than at least one opposite side of the tubing.

In one embodiment, shown in FIGS. 10 and 11, chamber 50 includes a first lobe 90 that is spaced apart from a second lobe 92 by a cylindrical section 94 when chamber 50 is in the second orientation. Lobes 90, 92 each have a first diameter when the member 40 is in the first orientation and a second diameter when member 40 is in the second orientation. Section 94 has a third diameter when member 40 is in the first orientation and the second orientation. In one embodiment, the third diameter is equal to the first diameter. In one embodiment, the third diameter is less than first diameter. In one embodiment, the third diameter is greater than the first diameter. In one embodiment, lobe 90 is inflatable independent of lobe 92 and vice versa. This configuration allows one of lobe 90 and lobe 92 have the second diameter when member 40 is in the second orientation and the other of lobe 90 and lobe 92 to have the first diameter when member 40 is in the second orientation. In one embodiment, a multiple lumen catheter is used such that each of lobe 90, 92 has access to its own lumen such that each of lobes 90, 92 can be selectively inflated by delivering fluid through a respective lumen.

An inner member 56 is disposed in passageway 48 and chamber 50 such that member 56 extends through openings 52, 54. Member 56 includes an inner surface 58 defining a second passageway 60. Passageway 60 extends parallel to axis A, has a cylindrical cross sectional configuration and a uniform diameter along the length of passageway 60. It is envisioned that passageway 60 may be disposed in orientations relative to axis A, such as, for example, transverse, perpendicular and/or other angular orientations such as acute, obtuse, co-axial and/or may be offset or staggered. It is further envisioned that all or only a portion of passageway 60 may be variously configured and dimensioned, such as, for example, planar, concave, convex, hemispherical, polygonal, irregular, uniform, non-uniform, staggered, tapered, consistent or variable, depending on the requirements of a particular application.

A first end 62 of member 56 includes a first circular opening 62 that is in communication with passageway 60 and a second end 64 of member 56 includes a second circular opening 64 that is in communication with chamber 50. Openings 52, 54 each extend parallel to axis A. As shown in FIG. 1, for example, end 62 extends through opening 52 and end 66 extends through opening 54. It is contemplated that opening 64 and/or opening 68 may be disposed at alternate orientations, relative to axis A, such as, for example, transverse, perpendicular and/or other angular orientations such as acute or obtuse, co-axial and/or may be offset or staggered. It is further envisioned that all or only a portion of opening 64 and/or opening 68 may have alternate cross section configurations, such as, for example, oval, oblong, triangular, square, polygonal, irregular, uniform, non-uniform, offset, staggered, and/or tapered.

In one embodiment where member 56 and member 40 are integrally formed, surface 46 of member 40 engages an outer surface of member 56 to fix member 40 relative to member 56. In embodiments that are not integrally formed, member 56 and member 40 may be connected through a y-adapter (not shown). In some embodiments, end 44 of member 40 is tethered to the outer surface of member 56 by a method of bonding or attachment, such as, for example, adhesive bonding, thermal bonding, laser bonding or RF bonding. In some embodiments, member 56 and member 40 are integrally formed.

A guide member 70, such as a guide wire or stylet extends through passageway 60 such that a first end 72 of member 58 extends through opening 64 and a second end 74 of member 58 extends through opening 68. End 74 is configured to penetrate tissue to fix system 30 relative to the anatomy of a patient, such as, for example, a bone in order to treat a bone disorder, such as, for example, a fracture. In some embodiments, member 70 is a metal wire. It is envisioned that member 70 may also comprise a molded plastic, a stainless steel material or Nitinol. In some embodiments, end 74 includes a trocar tip, a diamond tip, a threaded tip, or another pointed tip to facilitate insertion of end 74 into tissue, such as, for example, bone.

In some embodiments, as shown in FIGS. 18 and 19, system 30 includes a cannula 76 including a first inner surface 78 defining a first lumen 80 and a second inner surface 82 defining a second lumen 84. Lumen 80 is configured for disposal of member 40 and lumen 84 is configured for disposal of bone filler material or a tool configured to deliver a bone filler material. Lumen 80 is spaced apart from lumen 84 such that lumen 80 is not in communication with lumen 84. In some embodiments, lumens 80, 84 each have a cylindrical cross sectional configuration and a uniform diameter along the length of lumens 80, 84. It is envisioned that all or only a portion of lumen 80 and/or lumen 84 may be variously configured and dimensioned, such as, for example, planar, concave, convex, hemispherical, polygonal, irregular, uniform, non-uniform, staggered, tapered, consistent or variable, depending on the requirements of a particular application.

In one embodiment, a distal end of lumen 80 includes an aperture 86 having a first portion extending parallel to axis A and a second portion extending perpendicular to axis A and a distal end of lumen 84 includes an aperture 88 having a first portion extending parallel to axis A and a second portion extending perpendicular to axis A. This configuration allows member 40 to be deployed through lumen 80 in the direction shown by arrow D and/or the direction shown by arrow E and bone filler material to be delivered through lumen 84 in the direction shown by arrow F and/or the direction shown by arrow E.

In assembly, operation and use, system 30 is employed with a surgical procedure, such as, for a correction or treatment of bone fractures. It is contemplated that one or all of the components of system 30 can be delivered or implanted as a pre-assembled device or can be assembled in situ. System 30 may be completely or partially revised, removed or replaced. For example, system 30 can be employed with a surgical correction treatment of an applicable condition or injury of an affected portion of a patient, such as, for example, a fracture in an arm of a human patient, such as, for example, a Colles' fracture. It is envisioned that system 30 may also be used to treat other affected portions of the patient, such as, for example, a calcaneus bone, bones of the feet or hands, bones of the spine, bones of the arms and legs, etc.

In use, to treat a fracture, a medical practitioner obtains access to a surgical site including the fractured bone in any appropriate manner, such as through incision and retraction of tissues. In one embodiment, a drill is employed to remove bone tissue to provide access to a repair site. It is envisioned that system 30 can be used in any existing surgical method or technique including open surgery, mini-open surgery, minimally invasive surgery and percutaneous surgical implantation, whereby the fractured or injured bone is accessed through a mini-incision or sleeve that provides a protected passageway to the area. Once access to the surgical site is obtained, the particular surgical procedure can be performed for treating the injury or disorder. The configuration and dimension of system 30 is determined according to the configuration, dimension and location of a selected section of the bone fracture and the requirements of a particular application.

An incision is made in the body of a patient and a cutting instrument (not shown) creates a surgical pathway for implantation of components of system 30. This may include the use of a cannula or other device. A preparation instrument (not shown) can be employed to prepare tissue surfaces, as well as for aspiration and irrigation of a surgical region according to the requirements of a particular surgical application.

Member 70 is inserted through the surgical pathway and is disposed in tissue to fix member 70 relative to the surgical site. In some embodiments, end 74 of member 70 is configured to penetrate tissue. A cannula may be positioned over member 70 such that a distal end of the cannula is positioned in or adjacent the void. That is, after member 70 is fixed relative to the surgical site, member 40 is delivered to the surgical site by inserting member 70 through opening 68 and translating member 56 in the direction shown by arrow G such that member 70 is disposed in passageway 60 and end 72 extends through opening 64. This configuration allows member 40 to be orientated such that at least a portion of chamber 50 is positioned in or adjacent to the bone disorder. A material, such as, for example, saline, a contrast solution or compressed air may be inserted through passageway 48 and into chamber 50 to move chamber 50 from the first unexpanded orientation to the second expanded orientation. As chamber 50 moves from the first orientation to the second orientation, chamber 50 creates a void in or adjacent the bone defect.

After the void is formed in the bone, member 40 may be removed from the patient by moving member 40 in the direction shown by arrow GG relative to member 70 such that member 70 is no longer disposed in passageway 60. Bone filler material may then delivered through the cannula to deliver bone filler material into the void so as to at least partially fill the void and realign fragments of the fracture and/or elevate collapsed bone to its proper height. It is envisioned that the bone filler material may include autograft, allograft, demineralized bone matrix, mineral composites, blocks, granules and pellets and bone cement, such as, for example, polymethylmethacrylate (PMMA)-based material (Kyphon HV-R, ActivOs, ActivOs 10, Xpede), calcium phosphate (Skaffold, Norian, Hydroset, KyphOs FS) and calcium sulfate (OsteoSet), as well as other injectables. In some embodiments, a tool configured to deliver bone filler material may be delivered through the cannula to and the bone filler material delivered into the void so as to at least partially fill the void and realign fragments of the fracture and/or elevate collapsed bone to its proper height.

In embodiments that include cannula 76, member 40 may be positioned within lumen 80 for delivery to the surgical site. After chamber 50 expands to create the void in the bone, as discussed above, member 40 may be moved such that chamber 50 is spaced apart from the void. For example, chamber 50 may be retracted such that at least a portion of chamber 50 is positioned within passageway lumen 80. Cannula 76 is then rotated relative to the void such that a distal end of lumen 84 is positioned adjacent the void. Bone filler material or a tool configured to deliver bone filler material is then positioned within lumen 84 and the bone filler material delivered into the void so as to at least partially fill the void and realign fragments of the fracture and/or elevate collapsed bone to its proper height.

It is envisioned that chamber 50 may have a volume that is less than the volume of the bone defect when chamber 50 is in the second orientation. In such situations, member 70 may be inserted through the access path at a first trajectory relative to an axis defined by the bone, such as, for example, trajectory T1 shown in FIG. 3. Member 70 is then positioned within passageway 60 and member 56 and member 40 are moved relative to member 70 such that chamber 50 is positioned adjacent the bone defect. Chamber 50 is moved from the first orientation to the second orientation such that chamber 50 creates a first void extending parallel to trajectory T1. Member 40 may then removed from the patient through the access path or moved such that member 40 is spaced apart from the first void created by chamber 50. A second access path is made that is spaced apart from the access path. Member 70 may then be inserted through the second access path at a second trajectory T2 relative to the axis defined by the bone. Member 70 is then positioned within passageway 60 and member 56 and member 40 are moved relative to member 70 such that chamber 50 is positioned adjacent the first void. Chamber 50 is moved from the first orientation to the second orientation such that chamber 50 creates a second void extending parallel to trajectory T2. The second void at least partially overlaps the first void such that the combined volume of the first and second voids is greater than or equal to the volume of the bone defect. Bone filler material may then be delivered into the first and second voids to at least partially fill the first and second voids.

It is envisioned that chamber 50 may have a volume that is equal to or greater than the volume of the bone defect when chamber 50 is in the second orientation. In such situations, the shape or configuration of chamber 50, when chamber 50 is in the second orientation, may be selected according to the shape or configuration of the bone defect. For example, in one embodiment, in which the defect has a first end that has a width that is greater than a width of a second end of the defect, as shown in FIG. 6A, a member 40 with a chamber 50 having an asymmetrical configuration when chamber 50 is in the second orientation, as shown in FIG. 5, is selected such that chamber 50 will create a void that consumes the entire defect or substantially the entire defect when chamber is in the second orientation, as shown in FIG. 6B. In one embodiment, in which the defect has a substantially triangular configuration, as shown in FIG. 9A, a member with a chamber 50 having a triangular or substantially triangular configuration when chamber 50 is in the second orientation, as shown in FIG. 7, is selected such that chamber 50 will create a void that consumes the entire defect or substantially the entire defect when chamber 50 is in the second orientation, as shown in FIG. 9B.

In embodiments in which chamber 50 includes lobes 90, 92 connected by section 94, chamber 50 may be inserted in or adjacent to the bone defect, as shown in FIG. 14. Chamber 50 is then moved to the second orientation such that chamber 50 creates a void V having a first section V1 and a second section V2 connected by a cylindrical section V3, as shown in FIG. 12. In one embodiment, member 40 is moved relative to void V such that lobe 92 is positioned in void V1, as shown in FIG. 14. Chamber 50 is then moved from the first orientation to the second orientation such that lobe 90 creates a fourth void V4 that is connected to void V1 by a cylindrical section V5, as shown in FIG. 15. In embodiments in which lobes 90, 92 are independently inflatable, lobe 92 may be inflated prior to inflating lobe 90 to create void V4, such that lobe 92 is anchored in void V1 as lobe 90 is inflated to create void V4.

In one embodiment, member 40 is moved relative to void V such that lobe 90 is positioned in void V2, as shown in FIG. 16. Chamber 50 is then moved from the first orientation to the second orientation such that lobe 92 creates a fourth void V4 that is connected to void V2 by a cylindrical section V5, as shown in FIG. 17. In embodiments in which lobes 90, 92 are independently inflatable, lobe 90 may be inflated prior to inflating lobe 92 to create void V4, such that lobe 90 is anchored in void V2 as lobe 92 is inflated to create void V4.

In one embodiment, system 30 includes an agent, which may be disposed, packed or layered within, on or about the components and/or surfaces of the components of system 30. It is envisioned that the agent may include bone growth promoting material, such as, for example, bone graft to enhance fixation of the fixation elements with the bone in need of repair. It is further contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of system 30 can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto. For example, all or a portion of member 40, including chamber 50 can be modified or extended to accommodate particular formulations of balloon construction materials or fabrication techniques. Different balloon materials and surface coatings, or outer layers of different materials or surface coatings may also be applied to member 40 and/or chamber 50 to facilitate a smaller balloon profile, biocompatibility, lubrication as well as other properties. The embodiments above can also be modified so that some features of one embodiment are used with the features of another embodiment. One skilled in the art may find variations of these preferred embodiments, which, nevertheless, fall within the spirit of the present invention, whose scope is defined by the claims set forth below.

What is claimed is:

1. A device for performing a surgical procedure comprising:
an inflatable member extending along a longitudinal axis between a first end having a first opening and a second end having a second opening, an inner surface of the inflatable member defining a first passageway in the first end and a chamber in the second end, the first and second openings being in communication with the first passageway;
an inner member disposed in the first passageway and extending through the first and second openings, the inner member including an inner surface defining a second passageway, a first end of the inner member including a first opening and a second end of the inner member having a second opening, the first and second openings of the inner member being in communication with the second passageway, the second end of the inflatable member being bonded to an outer surface of the inner member; and
a guide member disposed in the second passageway and extending through the first and second openings of the inner member.

2. A device as recited in claim 1, further comprising an outer member comprising an inner surface defining a third passageway, wherein the third passageway comprises a first lumen having the inflatable member disposed therein and a second lumen having a bone filler material or a tool configured to deliver bone filler material disposed therein.

3. A device as recited in claim 2, wherein the first lumen is spaced apart from the second lumen.

4. A device as recited in claim 2, wherein at least one of the first and second lumens has an aperture extending parallel to the longitudinal axis.

5. A device as recited in claim 2, wherein at least one of the first and second lumens has an aperture extending transverse to the longitudinal axis.

6. A device as recited in claim 1, wherein the inflatable member is movable between a first orientation in which the chamber has a first diameter and a second orientation in which the chamber has a second diameter, the second diameter being greater than the first diameter.

7. A device as recited in claim 6, wherein the chamber has an asymmetrical configuration when the inflatable member is in the second orientation.

8. A device as recited in claim 6, wherein the chamber has a triangular cross sectional configuration when the inflatable member is in the second orientation.

9. A device as recited in claim 6, wherein the chamber includes a first lobe and a second lobe that is spaced apart from the first lobe, the first lobe being connected to the second lobe by a cylindrical section.

10. A device as recited in claim 9, wherein the first and second lobes have the first diameter when the inflatable member is in the first orientation and the second diameter when the inflatable member is in the second orientation and the cylindrical section has a third diameter when the inflatable member is in the first orientation and the second orientation, the third diameter being less than the first diameter.

11. A device as recited in claim 9, wherein first lobe is inflatable independent of the second lobe such that one of the first and second lobes have the second diameter when the inflatable member is in the second orientation and the other of the
first and second lobes have the first diameter when the inflatable member is in the second orientation.

12. A method for correction of a bone injury comprising:
providing a device comprising:
an inflatable member extending along a longitudinal axis between a first end having a first opening and a second end having a second opening, an inner surface of the inflatable member defining a first passageway in the first end and a chamber in the second end, the first and second openings being in communication with the first passageway,
an inner member disposed in the first passageway and extending through the first and second openings, the inner member including an inner surface defining a second passageway, a first end of the inner member including a first opening and a second end of the inner member having a second opening, the first and second openings of the inner member being in communication with the second passageway, the second end of the inflatable member being bonded to an outer surface of the inner member, and
a guide member disposed in the second passageway and extending through the first and second openings of the inner member;
creating an access path to a defect in a bone;
inserting the guide member through the access path such that a distal end of the guide member engages tissue to fix the guide member relative to the bone;
inserting the inner member over the guide member such that the chamber is positioned in or adjacent the defect; and
delivering a material through the first passageway and into the chamber to move the inflatable member from a first orientation in which the chamber has a first diameter to a second orientation in which the chamber has a second diameter, the second diameter being greater than the first diameter such that the chamber creates a void in or adjacent the defect when the inflatable member is in the second orientation.

13. The method of claim 12, wherein inserting the guide member through the access path comprises inserting the guide member through the access path at a first trajectory and the method further comprises:
moving inner member such that the chamber is spaced apart from the defect after delivering the material into the chamber;
inserting the guide member through the access path at a second trajectory;
inserting the inner member over the guide member such that at least a portion of the chamber is positioned in or adjacent the void; and
delivering the material through the first passageway and into the chamber to move the inflatable member from the first orientation to the second orientation such that the chamber creates a second void.

14. The method of claim 13, further comprising: removing inner member from the access path; and
delivering a bone filler material into at least one of the first and second voids to at least partially fill the first and second voids.

15. The method of claim 12, wherein:
the device further comprises an outer member comprising an inner surface defining a third passageway;
the third passageway comprises a first lumen having the inflatable member disposed therein and a second lumen having a bone filler material or a tool configured to deliver bone filler material disposed therein; and
the method further comprises:
inserting the outer member through the access path such that the first lumen is positioned adjacent the defect,
rotating the outer member relative to the defect such that the second lumen is positioned adjacent the defect, and
delivering a bone filler material through the second lumen and into the void to at least partially fill the void.

16. The method of claim 12, wherein the chamber includes a first lobe and a second lobe that is spaced apart from the first lobe by a cylindrical section, the first and second lobes have the first diameter when the inflatable member is in the first orientation and the second diameter when the inflatable member is in the second orientation, the cylindrical section has a third diameter when the inflatable member is in the first orientation and the second orientation, the third diameter being less than the first diameter, and the void includes a first void created by first lobe and a second void created by the second lobe.

17. The method of claim 16, wherein the first and second voids are spaced apart from one another by a cylindrical void created by the cylindrical section of the inflatable member.

18. The method of claim 17, further comprising:
moving the expandable member such that the second lobe is positioned in the first void; and
delivering a material into the chamber to move the inflatable member from the first orientation to the second orientation such that the second lobe creates a third void that is connected to the first void by a second cylindrical section.

19. The method of claim 17, further comprising:
moving the expandable member such that the first lobe is positioned in the second void; and
delivering a material into the chamber to move the inflatable member from the first orientation to the second orientation such that the first lobe creates a third void that is connected to the second void by a second cylindrical section.

20. A device for performing a surgical procedure comprising:
an inflatable member extending along a longitudinal axis between a first end having a first opening and a second end having a second opening, an inner surface of the inflatable member defining a first passageway in the first end and a chamber in the second end, the first and second openings being in communication with the first passageway;
an inner member disposed in the first passageway and extending through the first and second openings, the inner member including an inner surface defining a second passageway, a first end of the inner member including a first opening and a second end of the inner member having a second opening, the first and second openings of the inner member being in communication with the second passageway, the second end of the inflatable member being bonded to an outer surface of the inner member; and
a guide member disposed in the second passageway and extending through the first and second openings of the inner member; and
an outer member comprising an inner surface defining a third passageway, the third passageway comprising a first lumen having the inflatable member disposed therein and a second lumen having a bone filler material or a tool configured to deliver bone filler material disposed therein, the first lumen being spaced apart from the second lumen, at least one of the first and second lumens having an aperture including a first portion extending parallel to the longitudinal axis and a second portion extending transverse to the longitudinal axis,
wherein the inflatable member is movable between a first orientation in which the chamber has a first diameter and a second orientation in which the chamber has a second diameter, the second diameter being greater than the first diameter.
